(19) United States
(12) Patent Application Publication
(10) Pub. No.: US 2007/0072302 A1
(43) Pub. Date: Mar. 29, 2007

(54) Method for quantitatively determining the LDL particle number in a distribution of LDL cholesterol subfractions

(75) Inventors: Faith Clendenen, Oakland, CA (US); Christopher Boggess, Cambridge, MA (US); Frank Ruderman, San Rafael, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Berkeley HeartLab, Inc.

(21) Appl. No.: 11/522,591

(22) Filed: Sep. 18, 2006

Related U.S. Application Data
(60) Provisional application No. 60/722,051, filed on Sep. 29, 2005.
(60) Provisional application No. 60/721,825, filed on Sep. 29, 2005.
(60) Provisional application No. 60/721,665, filed on Sep. 29, 2005.
(60) Provisional application No. 60/721,756, filed on Sep. 29, 2005.
(60) Provisional application No. 60/721,617, filed on Sep. 29, 2005.

(51) Int. Cl. G01N 33/92

(52) U.S. Cl. 436/71

(57) ABSTRACT

The invention provides a method (e.g., a computer algorithm) for calculating a number of particles in a LDL subfraction. The method features the steps of: 1) measuring an initial distribution of LDL particles (e.g., a relative mass distribution) from a blood sample; 2) processing the initial distribution of LDL particles with a mathematical model to determine a modified distribution of LDL particles (e.g., a relative particle distribution); 3) determining a total LDL particle number value from a blood sample; and 4) analyzing both the modified distribution of particles and the total LDL particle number value to calculate the particle number value in an LDL subfraction.

### CROSS REFERENCES TO RELATED APPLICATION

This application claims the benefit of priority U.S. Provisional Patent Application Ser. No. 60/722,051, filed Sep. 29, 2005; U.S. Provisional Patent Application Ser. No. 60/721,825, filed Sep. 29, 2005; U.S. Provisional Patent Application Ser. No. 60/721,665, filed Sep. 29, 2005; U.S. Provisional Patent Application Ser. No. 60/721,756, filed Sep. 29, 2005; and U.S. Provisional Patent Application Ser. No. 60/721,617, filed Sep. 29, 2005; all of the above mentioned applications are incorporated herein by reference in their entirety.

### BACKGROUND

1. Field of the Invention

The present invention relates to a method for measuring and quantifying ‘subfractions’ of low-density lipoprotein cholesterol (referred to herein as ‘LDL’).

2. Description of the Related Art

Although mortality rates for cardiovascular disease (CVD) have been declining in recent years, this condition remains the primary cause of death and disability in the United States for both men and women. In total, nearly 70 million Americans have a form of CVD, which includes high blood pressure (approximately 50 million Americans), coronary heart disease (12.5 million), myocardial infarction (7.3 million), angina pectoris (6.4 million), stroke (4.5 million), congenital cardiovascular defects (1 million), and congestive heart failure (4.7 million). Atherosclerotic cardiovascular disease (ASCVD), a form of CVD, can cause hardening and narrowing of the arteries, which in turn restricts blood flow and impedes delivery of vital oxygen and nutrients to the heart. Progressive atherosclerosis can lead to coronary artery, cerebral vascular, and peripheral vascular disease, which in combination result in approximately 75% of all deaths attributed to CVD.

Various lipoprotein abnormalities, including elevated concentrations of LDL and increased small, dense LDL subfractions, are causally related to the onset of ASCVD. Over time these compounds contribute to a harmful formation and build-up of atherosclerotic plaque in an artery's inner walls, thereby restricting blood flow. The likelihood that a patient will develop ASCVD generally increases with increased levels of LDL cholesterol, which is often referred to as ‘bad cholesterol’. Conversely, high-density lipoprotein cholesterol (referred to herein as ‘HDL’) can function as a ‘cholesterol scavenger’ that binds cholesterol and transports it back to the liver for re-circulation or disposal. This process is called ‘reverse cholesterol transport’. A high level of HDL is therefore associated with a lower risk of heart disease and stroke, and thus HDL is typically referred to as ‘good cholesterol’.

A lipoprotein analysis (also called a lipoprotein profile or lipid panel) is a blood test that measures blood levels of LDL and HDL. One method for measuring HDL and LDL and their associated subfractions is described in U.S. Pat. No. 6,812,033, entitled ‘Method for identifying at-risk cardiovascular disease patients’. This patent, assigned to Berkeley HeartLab Inc. and incorporated herein by reference, describes a blood test based on gradient-gel electrophoresis (GGE). Gradient gels used in GGE are typically prepared with varying concentrations of acrylamide and can separate macromolecules according to mass with relatively high resolution compared to conventional electrophoretic gels. Using this technology, GGE determines subfractions of both HDL and LDL. For example, GGE can differentiate up to seven subfractions of LDL (referred to herein as LDL I, IIa, IIb, IIIa, IIIb, IVa, and IVb), and up to five subfractions of HDL (referred to herein as HDL 2b, 2a, 3a, 3b, 3c). Lipoprotein subfractions determined from GGE are also referred to as ‘sub-particles’, and correlate to results from a technique called analytic ultracentrifugation (AnUC), which is an established clinical research standard for lipoprotein subfractionation.

Elevated levels of LDL IVb, a subfraction containing the smallest LDL particles, have been reported to have an independent association with arteriographic progression; a combined distribution of LDL IIIa and LDL IIIb typically reflects the severity of this trait.

Apolipoproteins, such as apolipoprotein B100 (referred to herein as ‘Apo B’) are an essential part of lipid metabolism and are components of lipoproteins. Apo B and related compounds provide structural integrity to lipoproteins and protect hydrophobic lipids (i.e., non-water absorbing lipids) at their center. They are recognized by receptors found on the surface of many of the body's cells and help bind lipoproteins to those cells to allow the transfer, or uptake, of cholesterol and triglyceride from the lipoprotein into the cells. Elevated levels of Apo B correspond highly to elevated levels of LDL particles, and are also associated with an increased risk of coronary artery disease (CAD) and other cardiovascular diseases.

Each LDL cholesterol particle has an Apo B molecule, and thus to a first approximation LDL particle number and Apo B have a 1:1 correspondence. In addition, elevated levels of Apo B are considered markers for determining an individual's risk of developing CAD when conjunctively compared to elevated small, dense LDL particles. There may be some elevation of these values due to the inclusion of Apo B from very low density lipoproteins. However, this elevation is estimated to be less than 10% for triglyceride values of less than 200 mg/dL.

### SUMMARY OF THE INVENTION

In a first aspect, the invention provides a method (e.g., a computer algorithm) for calculating a number of particles in a LDL subfraction. The method features the steps of: 1) measuring an initial distribution of LDL particles (e.g. a relative mass distribution) from a blood sample; 2) processing the initial distribution of LDL particles with a mathematical model to determine a modified distribution (e.g., a relative particle distribution); 3) determining a total LDL value from a blood sample; and 4) analyzing both the modified distribution of particles and the total LDL particle number value to calculate the LDL particle number value in an LDL subfraction.

In a second aspect, the invention provides a system for monitoring a patient that includes: 1) a database that stores blood test information describing, e.g., a number of particles in an LDL subfraction; 2) a monitoring device comprising systems that monitor the patient's vital sign information; 3) a database that receives vital sign information from the monitoring device; and 4) an Internet-based system configured to receive, store, and display the blood test and vital sign information.

In embodiments, the mathematical model used in the algorithm analyzes at least one geometrical property of LDL particles (e.g., radius, diameter) within an LDL subfraction to determine a conversion factor. For example, the conversion factor can be derived from a ratio of surface areas for LDL particles within two subfractions. Typically the conversion factor is determined before any processing, and is a constant for all patients. Once determined, the algorithm uses the conversion factor to convert the relative mass distribution into a relative particle distribution, which is then used to quantify the LDL particle number in each LDL subfraction.

In a preferred embodiment, the method features the step of determining the total LDL particle number value from an Apo B value. In this case, for example, the Apo B value is measured from a blood sample during a separate blood test, and the LDL particle number value is determined by assuming the physiological 1:1 ratio between Apo B and the LDL particles. Once this assumption is made, the LDL particle number within each LDL subfraction can be calculated by multiplying the relative particle distribution by the total LDL particle number.

‘Blood test information’, as used herein, means information collected from one or more blood tests, such as a GGE-based test. In addition to a relative mass distribution of LDL particles, blood test information can include concentration, amounts, or any other information describing blood-borne compounds, including but not limited to total cholesterol, LDL (and subfraction distribution), HDL (and subfraction distribution), triglycerides, Apo B particle, lipoprotein (a), Apo E genotype, fibrinogen, folate, HbA_{1c}, C-reactive protein, homocysteine, glucose, insulin, and other compounds. ‘Vital sign information’, as used herein, means information collected from patient using a medical device, e.g., information that describes the patient's cardiovascular system. This information includes but is not limited to heart rate (measured at rest and during exercise), blood pressure (systolic, diastolic, and pulse pressure), blood pressure waveform, pulse oximetry, optical plethysmograph, electrical impedance plethysmograph, stroke volume, ECG and EKG, temperature, weight, percent body fat, and other properties.

The invention has many advantages, particularly because it provides a quantitized number of particles for each LDL subfraction, rather than just a relative percentage of a mass distribution of particles. For example, a patient's percent mass distribution of LDL particles may remain unchanged, increase or decrease over time in response to aggressive lipid-lowering therapy, especially when the patient's total cholesterol and LDL cholesterol are significantly lowered using a cholesterol-lowering compound (e.g., an HMG-coA reductase inhibitor, commonly called ‘statins’, such as Lipitor™). In contrast to a potential variable change in percent distribution of LDL subclasses, these therapies can lower the specific number of LDL particles within a given subfraction, as determined by the method of this invention. A physician may use this information, in turn, to develop a specific cardiac risk reduction program for the patient targeting a quantifiable lipid-lowering therapeutic response.

The patient's quantized number of particles in each LDL subfraction, taken alone or combined with other blood tests, may also be used in concert with an Internet-based disease-management system and a vital sign-monitoring device. This system can process information to help a patient comply with a personalized cardiovascular risk reduction program. For example, the system can provide personalized programs and their associated content to the patient through a messaging platform that sends information to a website, email address, wireless device, or monitoring device. Ultimately the Internet-based system, monitoring device, and messaging platform combine to form an interconnected, easy-to-use tool that can engage the patient in a disease-management program, encourage follow-on medical appointments, and build patient compliance. These factors, in turn, can help the patient lower their risk for certain medical conditions, such as CVD.

These and other advantages of the invention will be apparent from the following detailed description and from the claims.

### BRIEF DESCRIPTION OF THE DRAWINGS

FIG. 1 is a graph of a relative mass distribution of LDL particles separated into seven unique subfractions closely correlated by prior research to lipid subfractions originally defined by AnUC;

FIG. 2 is a flow chart describing an algorithm for calculating the number of LDL particles in each subfraction from the relative mass distribution of FIG. 1;

FIG. 3 is a graph of relative mass and relative number distributions of LDL particles; and

FIG. 4 is a high-level schematic view of an Internet-based system that collects and analyzes blood test information, such as a quantitative number of LDL particles within a subfraction as determined using the algorithm in FIG. 2.

### DETAILED DESCRIPTION OF THE INVENTION

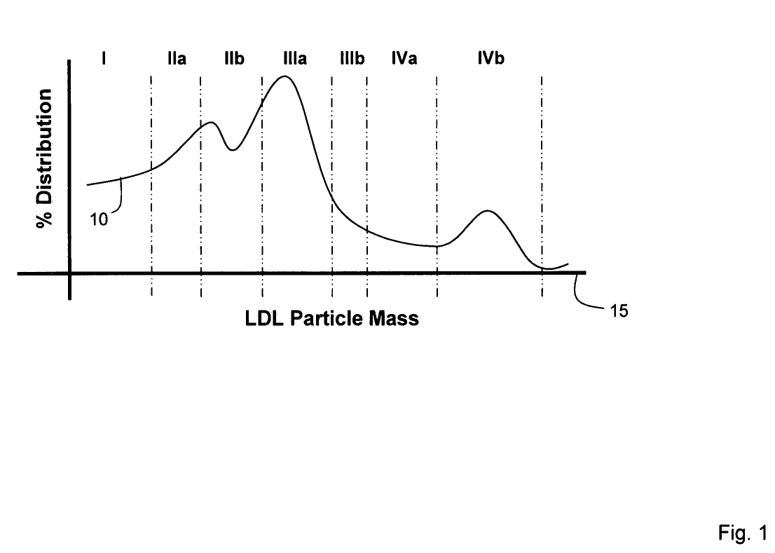

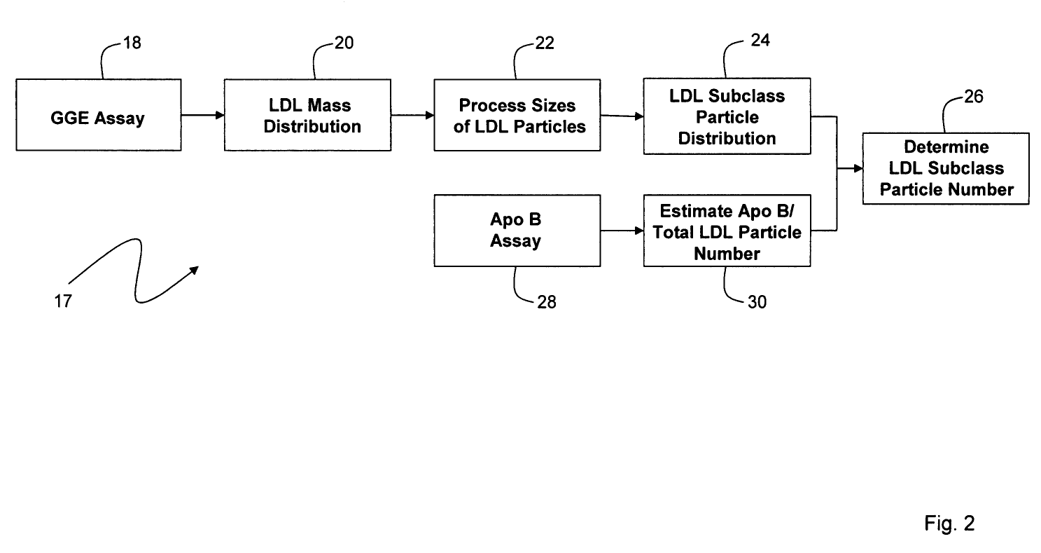

Referring to FIGS. 1 and 2, a conventional GGE process separates LDL particles into subfractions according to their mass, yielding a graph 15 that shows a relative mass distribution 10. The relative mass distribution 10 is sub-divided into seven LDL subfractions classified as I, IIa, IIb, IIIa, IIIb, IVa, IVb) that vary with particle size. Table 1, below, describes for each subfraction and corresponding region the: i) upper particle diameter; ii) lower particle diameter; iii) median diameter; and iv) mean radius. These values are well established and determined using separate studies, e.g., studies involving ultracentrifugation.

TABLE 1
LDL subfractions and their associated geometries

| | Upper | Lower | Median | Median |
|---|---|---|---|---|
| Subfraction | Diameter (Å) | Diameter (Å) | Diameter (Å) | Radius (Å) |
| I | 285.0 | 272.0 | 278.5 | 139.25 |
| IIa | 272.0 | 265.0 | 268.5 | 134.25 |
| IIb | 265.0 | 256.0 | 260.5 | 130.25 |
| IIIa | 256.0 | 247.0 | 251.5 | 125.75 |
| IIIb | 247.0 | 242.0 | 244.5 | 122.25 |
| IVa | 242.0 | 233.0 | 237.5 | 118.75 |
| IVb | 233.0 | 220.0 | 226.5 | 113.25 |

An algorithm 17, such as that shown in FIG. 2, quantitatively determines the number of LDL particles in each subfraction from the relative mass distribution 10. Analysis of a quantitative number of particles, as opposed to a relative mass distribution of particles, may help a medical professional design an effective, customized cardiac risk reduction program for the patient, such as that described in more detail below.

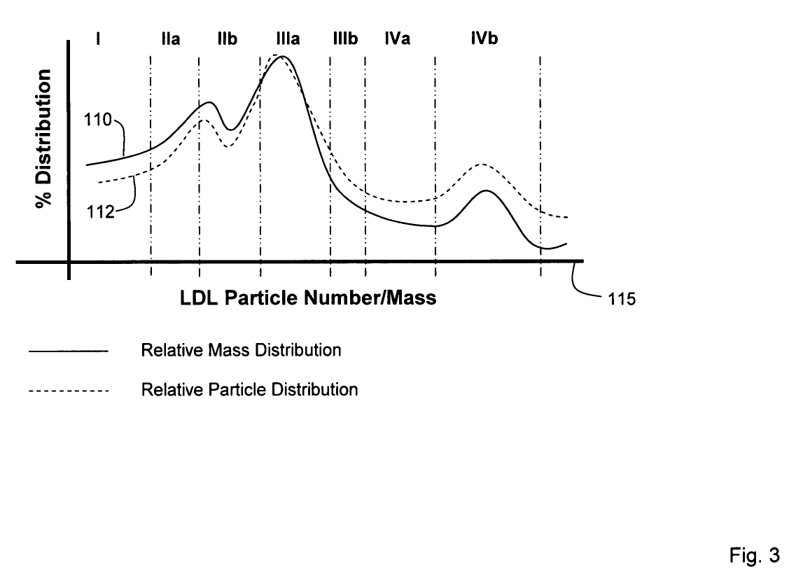

The algorithm 17 begins by processing inputs from a GGE assay (step 18) to generate a relative mass distribution of LDL particles (step 20), similar to that shown in FIG. 1. Such a GGE assay is described in U.S. Pat. No. 6,812,033, entitled ‘Method for identifying at risk cardiovascular disease patients’, the contents of which are incorporated herein by reference. The algorithm 17 processes the particle sizes corresponding to each subfraction (step 22) by assuming: i) all particles within the subfractions are spherical; and ii) the upper and lower diameters of particles in each subfraction are constant for all patients. This step of the algorithm 17 is described in more detail below with reference to FIG. 3. By processing the particle size, the algorithm 17 determines the relative surface area ratios for particles in each subfraction, and uses this value to convert the relative mass distribution into a relative particle distribution (step 24). The relative particle distribution describes the relative percentage of particles that correspond to each subfraction.

A separate branch of the algorithm 17 determines the total, quantitative number of LDL particles using an Apo B value measured with a separate assay (step 28). Once the Apo B value is determined, the algorithm 17 estimates the total number of LDL particles (step 30) by assuming a 1:1 relationship between these compounds. This relationship is well described in the following references, the contents of which are incorporated by reference: 1) Planella et al., ‘Calculation of LDL-Cholesterol by Using Apolipoprotein B for Classification of Nonchylomicronemic Dyslipemia’, Clinical Chemistry 43: 808-815, 1997; 2) Nauck et al., ‘Methods for Measurement of LDL-Cholesterol: A Critical Assessment of Direct Measurement by Homogeneous Assays Versus Calculation’, Clinical Chemistry 48:2; 236-54, 2002; 3) Berman et al., ‘Metabolism of Apo B and Apo C Apoproteins in Man: Kinetic Studies in Normal and Hyperlipoproteinemic Subjects’, Journal of Lipid Research 19:38-56, 1978; 4) Pease et al., ‘Regulation of Hepatic Apolipoprotein-B-Containing Lipoprotein Secretions’, Current Opinion in Lipidology 7:132-8, 1996; 5) Gaw et al., ‘Apolipoprotein B Metabolism in Primary and Secondary Hyperlipidemias’, Current Opinion on Lipidology 7:149-57, 1996; and 6) Mahley et al. ‘Plasma Lipoproteins and Apolipoprotein Structure and Function’, Journal of Lipid Research 25:1277-1294, 1984.

The algorithm then processes this value with the relative distribution of LDL particles (step 24) to quantitatively determine the number of LDL particles in each sub-fraction (step 26).

After determining this profile, the algorithm can integrate with other software systems for disease management, such as those described below and in the following references, the contents of which are incorporated herein by reference: 1) INTERNET-BASED SYSTEM FOR MONITORING LIPID, VITAL-SIGN, AND EXERCISE INFORMATION FROM A PATIENT (filed Sep. 29, 2005); 2) INTERNET-BASED PATIENT-MONITORING SYSTEM FEATURING INTERACTIVE MESSAGING ENGINE (filed Sep. 29, 2005); 3) APOLIPOPROTIEN E GENOTYPING AND ACCOMPANYING INTERNET-BASED HEALTH MANAGEMENT SYSTEM (attached hereto); and 4) INTERNET-BASED HEALTH MANAGEMENT SYSTEM FOR IDENTIFYING AND MINIMIZING RISK FACTORS CONTRIBUTING TO METABOLIC SYNDROME (filed Sep. 29, 2005). Copies which are attached and are part of this disclosure.

The algorithm described in FIG. 2 requires a calculation to determine the relative particle distribution from the relative mass distribution of LDL particles. To make this calculation, the algorithm assumes each LDL particle is spherical, and thus the particle's average surface area (SA) is:
SA=4πr²
Using the values from Table 1, above, the relative proportion of the surface areas of LDL I and LDL IVb is:
4π(139.25)²/4π(113.25)²=1.512

This means LDL particles in subfraction I have 1.512 times the surface area of particles in subfraction IVb. The relative surface area ratios between LDL I and other LDL particles shown in Table 1 can be calculated with this same methodology:

TABLE 2
ratio and inverse of ratio of surface areas
of LDL IVb and other LDL subfractions

| | Ratio with | Inverse of |
|---|---|---|
| Subfraction | Subfraction IVb | Ratio |
| I | 1.512 | 0.661 |
| IIa | 1.405 | 0.712 |
| IIb | 1.323 | 0.756 |
| IIIa | 1.233 | 0.811 |
| IIIb | 1.165 | 0.858 |
| IVa | 1.099 | 0.910 |
| IVb | 1.000 | 1.000 |

The inverse of the ratios shown in Table 2 yields a factor that converts the relative mass distribution of LDL particles to a corresponding relative particle distribution. For example, assume a relative mass distribution featuring 50% of the relatively large LDL I particles and 50% of the relatively small LDL IVb particles, as measured with a conventional GGE-based assay: for every 10 LDL IVb particles there are 6.61 LDL I particles. Using this same methodology and the factors in Table 2, the entire relative number distribution of LDL particles can be calculated from the relative mass distribution measured from a conventional GGE assay. In the above example, for instance, the relative mass distribution of 50% LDL IVb particles and 50% LDL I particles converts into a relative particle distribution of 60.2% LDL IVb particles (% of 10/(10+6.61)) and 39.8% LDL I particles (% of 6.61/(10+6.61)). Thus, in comparison to their relative mass distribution, the relative number of larger particles (e.g., LDL I particles) decreases, while the relative number of smaller particles (e.g., LDL IVb particles) increases.

The algorithm measures the quantitative number of particles in each subfraction by multiplying percentages from the relative number distribution by the total number of LDL particles, determined from the Apo B value as described above.

FIG. 3 shows a schematic drawing comparing for LDL a relative mass distribution 110 (measured with a GGE assay) to a relative particle distribution 115 (calculated with the above-described algorithm). As indicated above, the relative proportions of subfractions within the two distributions are different because of the variation in size of the particles within the subfractions. Specifically, the particle distribution of the larger particles (e.g., LDL I, IIa, and IIb) decreases relative to a mass distribution of the same particles. And conversely a particle distribution of the smaller particles (e.g., LDL IIIa, IIIb, IVa, and IVb) increases relative to a mass distribution of the same particles.

Studies in the literature indicate that careful analysis of a patient's LDL subfractions can determine their risk for CAD. For this reason, in embodiments the invention provides an Internet-based disease-management system that analyzes the number of LDL particles measured in each subfraction, and in response designs a customized cardiac risk reduction program for the patient. The system can also provide personalized programs and their associated content to the patient through a messaging platform that sends information to a website, email address, wireless device, or monitoring device. Ultimately the disease-management system and messaging platform combine to form an interconnected, easy-to-use tool that can engage the patient, encourage follow-on medical appointments, and build patient compliance. These factors, in turn, can help the patient lower their risk for certain medical conditions, such as CVD.

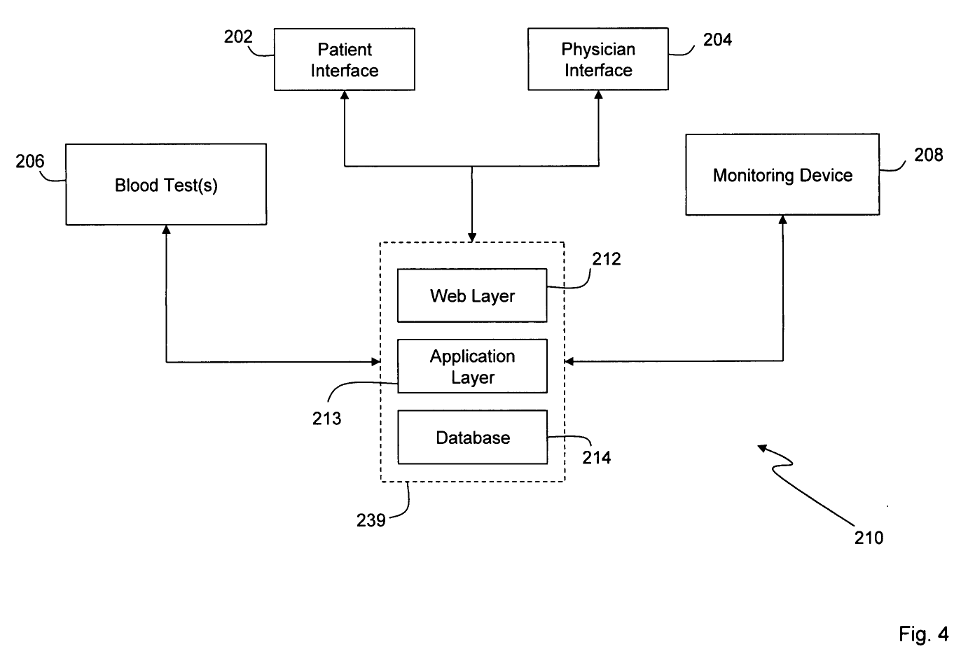

FIG. 4, for example, shows an Internet-based system 210 according to the invention that collects blood test information, such as information describing LDL cholesterol subfractions, from one or more blood tests 206, and vital sign information (e.g., blood pressure, heart rate, pulse oximetry, and ECG information) from a monitoring device 208. Such a system is described, for example, in INTERNET-BASED SYSTEM FOR MONITORING LIPID, VITAL-SIGN, AND EXERCISE INFORMATION FROM A PATIENT (filed Sep. 29, 2005), the contents of which were previously incorporated herein by reference. The Internet-based system 210 features a web application 239 that manages software for a database layer 214, application layer 213, and interface layer 212 for, respectively, storing, processing, and displaying information. The web application 239 renders information from a single patient on a patient interface 202, and information from a group of patients on a physician interface 204. More specifically, within the web application 239, the application layer 213 features information-processing algorithms that analyze the blood test and vital sign information stored in the database layer 214. Analysis of this information can yield a metabolic and cardiovascular risk profile that, in turn, can help the patient comply with a physician-directed cardiovascular risk reduction program. Specifically, based on this analysis, the interface layer 212 may render one or more web pages that describe a personalized program that includes reports and recommendations for diet, exercise, and lifestyle changes, along with content such as “heart-healthy” food recipes and news and reference articles. These web pages are available on both the patient 202 and physician 204 interfaces.

Other embodiments are also within the scope of the invention. For example, the blood test and analysis method for determining the number of particles in each LDL cholesterol subfraction can be combined with other blood tests. In other embodiments, mathematical algorithms other than those described above can be used to analyze the LDL particles to convert a relative mass distribution into a relative particle distribution. In other embodiments, the total LDL value is measured directly, as opposed to being calculated from an Apo B value.

In still other embodiments, the web pages used to display information can take many different forms, as can the manner in which the data are displayed. Different web pages may be designed and accessed depending on the end-user. As described above, individual users have access to web pages that only chart their vital sign data (i.e., the patient interface), while organizations that support a large number of patients (e.g., doctor's offices and/or hospitals) have access to web pages that contain data from a group of patients (i.e., the physician interface). Other interfaces can also be used with the web site, such as interfaces used for: hospitals, insurance companies, members of a particular company, clinical trials for pharmaceutical companies, and e-commerce purposes. Vital sign information displayed on these web pages, for example, can be sorted and analyzed depending on the patient's medical history, age, sex, medical condition, and geographic location.

The web pages also support a wide range of algorithms that can be used to analyze data once it is extracted from the blood test information. For example, the above-mentioned text message or email can be sent out as an ‘alert’ in response to vital sign or blood test information indicating a medical condition that requires immediate attention. Alternatively, the message could be sent out when a data parameter (e.g. blood pressure, heart rate) exceeded a predetermined value. In some cases, multiple parameters can be analyzed simultaneously to generate an alert message. In general, an alert message can be sent out after analyzing one or more data parameters using any type of algorithm.

The system can also include a messaging platform that generates messages which include patient-specific content (e.g., treatment plans, diet recommendations, educational content) that helps drive the patient's compliance in a disease-management program (e.g. a cardiovascular risk reduction program), motivate the patient to meet predetermined goals and milestones, and encourage the patient to schedule follow-on medical appointments. Such a messaging system is described in a co-pending application entitled ‘INTERNET-BASED PATIENT-MONITORING SYSTEM FEATURING INTERACTIVE MESSAGING ENGINE’ (filed Sep. 29, 2005) the contents of which have been previously incorporated herein by reference.

In certain embodiments, the above-described can be used to characterize a wide range of maladies, such as diabetes, heart disease, congestive heart failure, sleep apnea and other sleep disorders, asthma, heart attack and other cardiac conditions, stroke, Alzheimer's disease, and hypertension.

Still other embodiments are within the scope of the following claims.

I claim as my invention:

1. A method for calculating a number of particles in an LDL cholesterol subfraction, comprising the steps of:
1) measuring an initial distribution of LDL particles from a blood sample;
2) processing the initial distribution of LDL particles with a mathematical model to determine a modified distribution of LDL particles;
3) determining a total LDL particle number value from a blood sample; and
4) analyzing both the modified distribution of particles and the total LDL particle number value to calculate the LDL particle number in an LDL subfraction.

2. The method of claim 1, wherein the initial distribution of LDL particles is a relative mass distribution.

3. The method of claim 2, wherein the processing step further comprises processing the relative mass distribution with a mathematical model that converts it to a relative particle distribution.

4. The method of claim 3, wherein the mathematical model analyzes at least one geometrical property of LDL particles within an LDL subfraction to determine a conversion factor.

5. The method of claim 4, wherein the geometrical property describes a size of the particle, and the conversion factor is derived from a ratio of a first surface area of a LDL particle within a first LDL subfraction, and second surface area of a LDL particle within a second LDL subfraction.

6. The method of claim 1, wherein the processing step further comprises processing the initial distribution of LDL particles with a mathematical model to determine a relative LDL particle distribution.

7. The method of claim 6, wherein the processing further comprises converting a relative mass distribution of LDL particles into a relative LDL particle distribution with the mathematical model.

8. The method of claim 1, wherein the determining step further comprises determining the total LDL particle number value from an Apo B value or a derivative thereof.

9. The method of claim 8, further comprising the steps of: 1) measuring an Apo B value or a derivative thereof from a blood sample; and 2) assuming a ratio between Apo B and the total LDL particle number value.

10. The method of claim 9, further comprising the step of assuming a 1:1 ratio between Apo B and LDL particles.

11. The method of claim 1, wherein the measuring step further comprises measuring an initial distribution of LDL particles from a blood sample using a GGE-based assay.

12. The method of claim 1, wherein the measuring step further comprises measuring an initial distribution of LDL particles from an ultracentrifugation assay.

13. A method for calculating a particle number in an LDL subfraction, comprising the steps of:
1) measuring a relative mass distribution of LDL particles from a blood sample;
2) processing the relative mass distribution of LDL particles with a mathematical model to determine a relative particle distribution of LDL particles;
3) determining a total LDL particle number value from a blood sample; and
4) analyzing both the relative particle distribution and the total LDL particle number value to calculate the LDL particle number in an LDL subfraction.

14. The method of claim 13, wherein the mathematical model analyzes at least one geometrical property of LDL particles within an LDL subfraction to determine a conversion factor.

15. The method of claim 14, wherein the geometrical property is a size of the particle, and the conversion factor is derived from a ratio of a first surface area of a LDL particle within a first LDL subfraction, and second surface area of a LDL particle within a second LDL subfraction.

16. The method of claim 13, wherein the determining step further comprises determining the total LDL particle number value from an Apo B value or a derivative thereof.

17. The method of claim 16, further comprising the steps of: 1) measuring an Apo B value or a derivative thereof from a blood sample; and 2) assuming a ratio between Apo B and a total number of LDL particles.

18. The method of claim 17, further comprising the step of assuming a 1:1 ratio between Apo B and the total number of LDL particles.

19. A system for monitoring a patient, comprising:
a database that stores blood test information describing a particle number for an LDL subfraction;
a monitoring device comprising systems that monitor the patient's vital sign information;
a database that receives vital sign and exercise information from the monitoring device; and
an Internet-based system configured to receive, store, and display the blood test, vital sign, and exercise information.
